(19) United States
(12) Patent Application Publication
(10) Pub. No.: US 2019/0290326 A1
(43) Pub. Date: Sep. 26, 2019

(54) TROCAR WITH DUAL-PROTECTION SHEILD

(71) Applicant: 5RMED TECHNOLOGY (CHENGDU) CO., LTD, Chengdu (CN)

(72) Inventor: Moshu ZHU, Chengdu (CN)

(21) Appl. No.: 16/435,378

(22) Filed: Jun. 7, 2019

Related U.S. Application Data
(63) Continuation of application No. PCT/CN2017/113729, filed on Nov. 30, 2017.

(30) Foreign Application Priority Data
Dec. 9, 2016 (CN) 201611125378.X

(51) Int. Cl. A61B 17/34

(52) U.S. Cl. CPC A61B 17/3496; A61B 2017/0042; A61B 17/3423

(57) ABSTRACT

The application relates to a trocar with dual-protection shield comprises an obturator and a cannula, said cannula including a hollow sleeve, which includes a sleeve distal-end and a sleeve-lip, said obturator including a handle, the distal-end portion, and a shaft therebetween. When the trocar is in operation, the obturator penetrates through the cannula into the body wall to establish a penetration channel. The shaft includes a fastened shaft and a movable shaft; the fastened protection shield comprises a base of the fastened shield and a slant distal-end of the fastened shield connected thereto, the slant distal-end including a distal shaft-aperture, which penetrates the slant distal-end to form a fastened-shield-lip; the fastened protection shield is connected with the fastened shaft, the working-blade extending toward the proximal end and connected with a fastened shaft or handle, the working-blade extending toward the distal end beyond the fastened-shield-lip.

### CROSS-REFERENCE TO RELATED APPLICATIONS

This application is a continuation of International Patent Application No. PCT/CN2017/113729, filed on Nov. 30, 2017, which claims priority to Chinese Patent Application No. 201611125378.X, filed on Dec. 9, 2016. The content of the aforementioned applications, including any intervening amendments thereto, are incorporated herein by reference.

### TECHNICAL FIELD

The present application relates to a minimally invasive surgical instrument, and in particular, to a trocar obturator.

### BACKGROUND

A trocar is a surgical instrument that is used to establish an artificial access in minimally invasive surgery (especially in rigid endoscopy). A trocar assembly generally comprise in general a cannula and an obturator. The general clinical use is as follows: firstly cut a small incision on the patient's skin, and then pass the obturator through the cannula, the distal end of the obturator exceeds the distal end of the cannula, and then through the skin opening penetrating the body wall into the body cavity.

During penetration, the surgeon holds the trocar and applies a large penetration force to overcome the resistance to penetrating and cutting the tissue, as well as the resistance to expansion and swelling of the tissue. The distal end of the obturator usually contains a sharp blade that helps reduce the penetration force and the cutting-tissue force. At the moment of penetrating the body wall, the resistance suddenly disappears, and the surgeon may not be able to stop applying force or due to inertia, so the blade may accidentally damage the interior tissue of the patient. Therefore, the obturator usually includes a selective-axial-moved protection shield and an automatic lock device, which is called an automatic protection obturator with blade (hereinafter referred to as a protection obturator). Said protection obturator is possessed of a lock state and a release state: in the released state, the protection shield may be retracted from the distal end to the proximal end and expose the blade; in the protective state, the protection shield cannot be retracted from the distal end to the proximal end and the blade is covered by the protection shield. At the moment of penetrating the body wall, the automatic lock device is triggered almost simultaneously, and the protection shield is moved almost instantaneously to the distal end covering blade and locked, thereby preventing the blade from being exposed to cause damage. At the moment of penetrating the body wall, the protection shield is moved almost instantaneously to the distal end covering blade and locked, thereby preventing the blade from being exposed to cause damage.

When the blade and the protection shield of the obturator penetrate the body wall, the process of covering the blade and locked by the protection shield from the proximal end to the distal end is delayed, due to the resistance between the muscle and the tissue of the body wall and the protection shield. FIG. 1 illustrates a schematic view of a protective trocar 10 that penetrates into the body wall in the conventional clinical application. The protective trocar 10 comprises a blade 20, a protection shield 30, a sleeve distal-end 40 and a reset spring 50 (not shown), the protection shield 30 including a conical tip 32, a cylindrical end 34 and a groove 36, the sleeve distal-end 40 including a slanted sleeve-lip 41. The process by which the trocar 10 penetrates into the body wall primarily includes the blade 20 cutting the muscles and tissue, the conical tip 32 expanding the incision, and the sleeve distal-end 40 expanding the incision. Referring to FIG. 1, when the blade 20 and the conical tip 32 completely penetrate through the body wall into the body cavity, the blade 20 has been exposed outside of the protection shield 30, with a great risk of accidentally damaging the interior organs or tissue. Ideally, the reset force of the reset spring 50 should immediately drive the protection shield 30 to be moved from the proximal end to the distal end and cover the blade 20; however, since the cylindrical end 34 of the protection shield 30 is exposed outside the slanted sleeve-lip 41, there is a large friction between the cylindrical end 34 and the muscle tissue. The protection shield 30 cannot cover the blade 20 when its frictional force is greater than the reset force of the protection shield 30. Increasing the reset force can solve this problem, but increasing the reset force inevitably increases the resistance of the penetration process, thereby increasing the overall penetration force. So far, there have been no detailed studies and solutions to this problem in the prior art disclosed.

For reducing the risk of damage to interior organs, in the clinical application, when the surgeon holds trocar for penetrating operation, the manner of penetrating into the body is rotating back and forth in a small range instead of a simple linear motion. The round-trip rotary manner is beneficial for tearing and swelling muscle tissue, and for controlling the penetration speed and reducing the aforementioned inertia effect. While in this the round-trip rotary manner, the blade of the protection obturator rotates back and forth and cuts muscle tissue, resulting in irregular wounds, thereby additionally increasing the damage to the patient, and increasing the occurrence probability of incision hernia complication.

Studies have shown that the obturator without blade (hereinafter referred to as the bladeless obturator) is beneficial for reducing damage to the patient. When penetrating the body wall with the bladeless obturator, the distal end of the bladeless obturator penetrates the muscle and tissue due to the absence of a sharp blade, separates the muscle fibers and swells the wound until the obturator and the cannula assembly passing through the body wall. Compared with the protective obturator, the bladeless obturator reduces the cutting damage to the muscle tissue, helps the postoperative recovery, and helps reducing the probability of incision hernia complication. It is generally concluded that the use of a bladeless obturator is less harmful to the patient than the use of a blade (protection) obturator. However, when the obturator is used for penetration, the penetration force is generally larger than which of protective obturator, so it is more difficult to control, and the risk of damage to organs and tissues for the patient is increased.

In order to solve the problem or several problems descried above, the present invention proposes a trocar (obturator) with dual-protection shield.

### SUMMARY

In conclusion, one object of the invention is to provide a trocar (obturator) with dual-protection shield.

In one aspect of the invention, an trocar (obturator) with dual-protection shield comprises an obturator and a cannula, said cannula including a hollow sleeve, which includes a sleeve distal-end and a sleeve-lip, said obturator including a handle, the distal-end portion, and a shaft therebetween. When the trocar is in operation, the obturator penetrates through the cannula into the body wall to establish a penetration channel.

The distal-end portion includes a fastened protection shield, a working-blade and an adjustable (movable) protection shield. The shaft includes a fastened shaft and a movable shaft. The fastened protection shield comprises a base of the fastened shield and a slant distal-end of the fastened shield connected thereto, the slant distal-end including a distal shaft-aperture, which penetrates the slant distal-end to form a fastened-shield-lip. The fastened protection shield is connected with the fastened shaft, the working-blade extending toward the proximal end and connected with a fastened shaft or handle, the working-blade extending toward the distal end beyond the fastened-shield-lip. When passing the obturator through the cannula for the penetration, the slant distal-end of the fastened shield is exposed outside the sleeve-lip, and the movable protection shield comprises a base of the movable shield and the slant distal-end of the movable shield connected thereto. The distal-end portion includes an operation mode and a protection mode, in the operation mode, the movable protection shield is movable from the distal end to the proximal end to expose the working-blade, and in the protection mode, and the working-blade is covered by the movable protection shield. The handle includes a lock mechanism to effect mutual switching between the working mode and the protection mode.

In one embodiment, the base of the movable shield comprises a straight cylinder formed by the straight cylinder-surface, the shape and size of the straight cylinder matching the distal axial-aperture. Optionally, the size of the movable-shield-base conforms to the following equation:

0.25A₀≤A≤0.72A₀

wherein:

A=cross-sectional area of the straight cylinder

A₀=the maximum cross-sectional area of the distal-end portion

In one embodiment, the working-blade comprises a metal blade and a metal blade and metal blade-edge. In another embodiment, the working-blade comprises a plastic blade and a plastic blade-edge.

In another aspect of the invention, the handle of the obturator includes a lock mechanism, which comprises at least a lock, a release, and a trigger. The lock mechanism includes an initial lock state, a release state, a trigger state, and a reset lock state. In the initial locked state, the distal-end portion is in a protection mode; in the release state and the trigger state, the distal portion is in the operation mode; in the reset lock state, the distal portion transitions from the operation mode to the protection mode.

### BRIEF DESCRIPTION OF THE DRAWINGS

A more complete appreciation of this invention, and many of the attendant advantages thereof will be readily apparent as the same becomes better understood by reference to the following detailed description, where:

FIG. 1 is a schematic view of the trocar penetrating into the body wall in the prior art;

FIG. 2 is a 3D perspective view of the trocar assembly;

FIG. 3 is a 3D perspective view of the obturator in the first embodiment of the invention;

FIG. 4 is a 3D perspective exploded-view of the obturator in FIG. 3;

FIG. 5 is a projection view of the fastened-portion in FIG. 4;

FIG. 6 is a cross-section view taken along line 6-6 of FIG. 5;

FIG. 7 is a projection view of the movable protection shield in FIG. 4;

FIG. 8 is a cross-section view taken along line 8-8 of FIG. 7;

FIG. 9 is a perspective view of the lock member in FIG. 4;

FIG. 10 is a longitudinal cross-sectional view of the vertical working-blade in FIG. 4;

FIG. 11 is a partial 3D perspective view of the handle in FIG. 10;

FIG. 12 is a schematic view of the obturator in the release state in FIG. 10;

FIG. 13 is a schematic view of the obturator in the trigger state in FIG. 10;

FIG. 14 is a simulated schematic view of the body wall in FIG. 13;

FIG. 15 is a simulated schematic view of the movable protection shield penetrating through the cannula into the body wall;

FIG. 16 is a 3D perspective enlarged-view of the movable protection shield of FIG. 7;

FIG. 16A: shows a sectional view along line 16A-16A in FIG. 16;

FIG. 17 is a 3D perspective enlarged-view of another movable protection shield;

FIG. 17A: shows a sectional view along line 17A-17A in FIG. 17;

FIG. 18 is a 3D perspective enlarged-view of another movable protection shield;

FIG. 18A: shows a sectional view along line 18A-18A in FIG. 18;

FIG. 19 is a 3D perspective view of the obturator in the second embodiment of the invention;

FIG. 20 is a 3D perspective exploded-view of the obturator in FIG. 19;

FIG. 21 is a 3D perspective view of the movable protection shield in FIG. 20.

FIG. 22 is a 3D perspective view of the fastened protection shield in FIG. 20.

FIG. 23 is a partial 3D cross-section view of the distal-portion in FIG. 19;

FIG. 24 is a schematic view of the obturator in the release state in FIG. 19;

FIG. 25 is a schematic view of the obturator in the trigger state in FIG. 19;

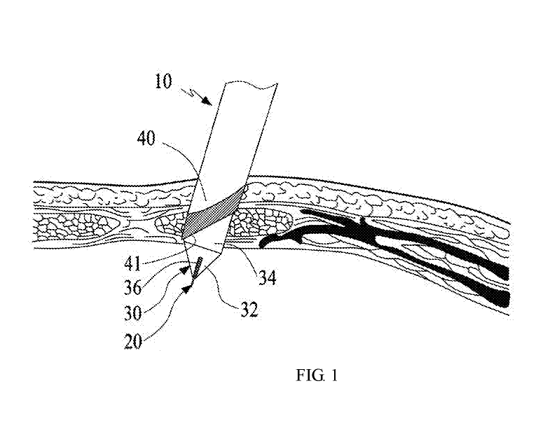

In all views, the same referred number shows the same element or assembly.

### DETAILED DESCRIPTION OF EMBODIMENTS

Embodiments of the invention are disclosed herein, however, it should be understood that the disclosed embodiments are merely examples of the invention, which may be implemented in different ways. Therefore, the invention is not intended to be limited to the detail shown, rather, it is only considered as the basis of the claims and the basis for teaching those skilled in the art how to use the invention.

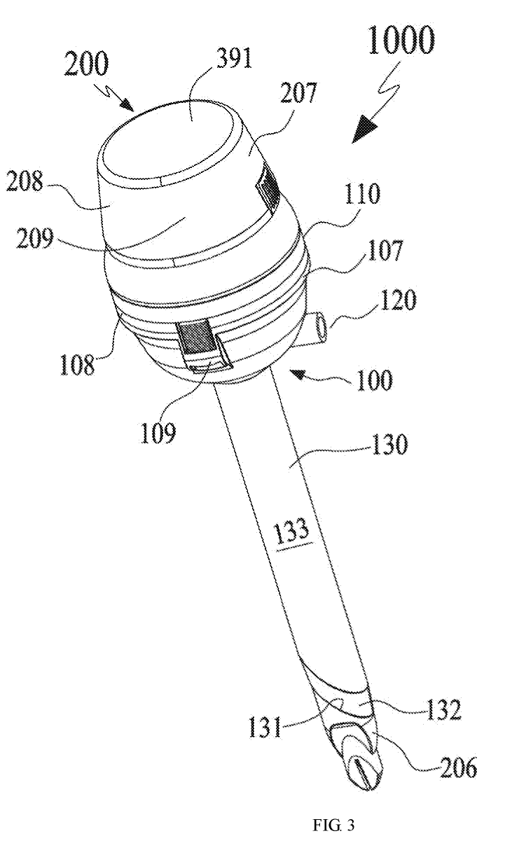

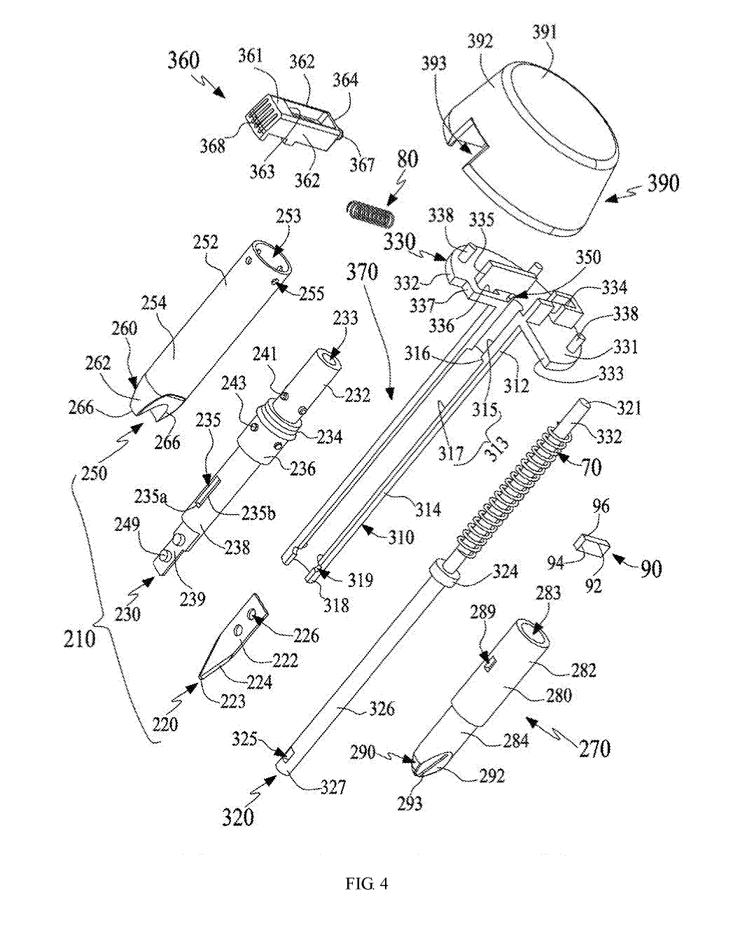

FIG. 3-4 illustrate the structure of the trocar 1000. A trocar 1000 comprises the cannula 100 and the obturator 200, the cannula 100 including a seal housing 110, a valve 120, and a sleeve 130. The seal housing 110 comprises a cannula top-surface 111 (not shown) and a hollow aperture 113 (not shown). In general, the duckbill seal (also known as closure valve) and a seal membrane (also known as instrument seal) are in turn secured in the seal housing 110 from the distal end to the proximal end. Said duckbill seal normally does not provide sealing for the inserted instrument, but automatically closing and forming a seal when the instrument is removed; said seal membrane accomplishes a gas-tight seal against the instrument when it is inserted. The sleeve 130 includes an open sleeve-distal-end 132 and an elongate shaft 133 that connected with the seal housing 110, the sleeve-distal-end 132 including a sleeve-lip 131. The obturator 200 is composed of a handle 202, a shaft 204 and the distal-end portion 206. The handle includes a top-wall 391 and an under-surface 333.

Referring to FIG. 3-4, the obturator 200 passes through the cannula 100, and the cannula top-surface 111 is connected with the handle under-surface 333. One side of the cannula 100 that limits the valve 120 is the front surface 107, an opposite side of which is the back surface 108, both sides of which is the side surfaces 109. The front surface 207, the back surface 208, and the left and right side surfaces 209 of the obturator are limited in accordance with the positional relationship when the obturator 200 is mated with the cannula 100. When the penetration is performed, the doctor grips the seal housing 110, and the palm rests against the top-wall 221 of the handle, continuously applying the penetration force to penetrate the patient's body wall. Once penetrated into the body cavity, the obturator is removed, and the cannula will be left as access for the instrument get in/out of the body cavity. For convenience of description, in the following the portion close to the surgeon is limited as the proximal end, and the portion far from the surgeon is limited as the distal end. The central axis of the obturator shaft 204 is limited as the axis 201. The direction substantially parallel to the axis 201 is referred to be the axial direction and the direction substantially perpendicular to the axis 201 is referred to the transverse direction.

FIGS. 4-10 show detailed depiction the first embodiment in the invention, the composition and assembly relationship of the blade auto-protection obturator 200. Referring to FIGS. 4-5, the distal portion 206 of the obturator 200 comprises a fastened distal-portion 210 and a movable distal-portion 270. The fastened distal-portion 210 includes a working-blade 220, a handle 230 and a fastened protection shield 250, and the movable distal-portion 270 includes a movable protection shield 280.

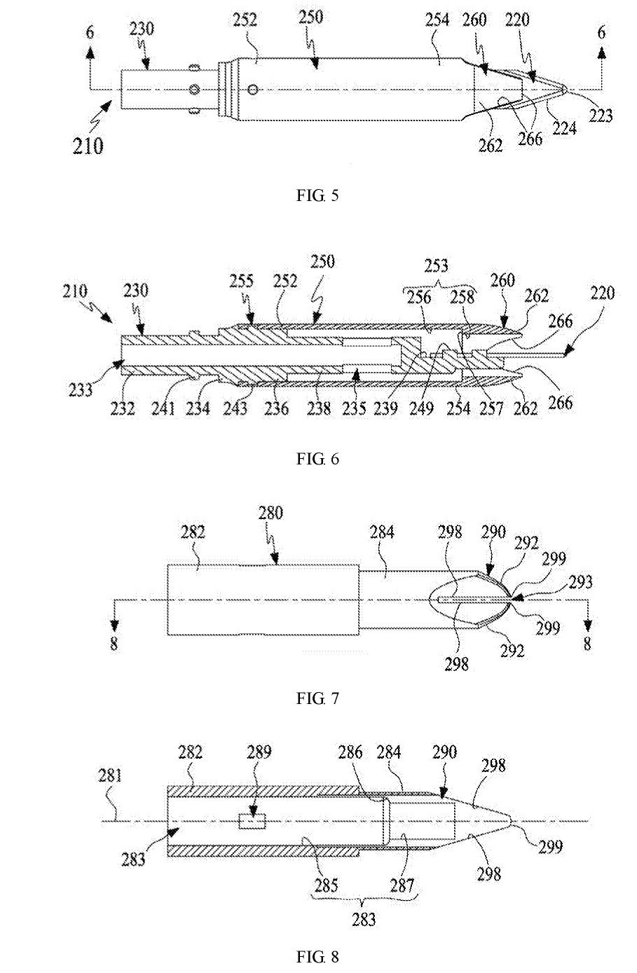

Referring to FIG. 5-6, the working-blade 220 includes a flat blade-body 222, a blade-tip 223 and two blade-edges 224 forming an acute angle with each other. Those skilled in the art can appreciate that the working-blade 220 and the handle 230 can be secured together by a variety of well-known joining techniques, such as bonding, welding, mechanical securing, and so on. In the present embodiment, the handle 230 includes a retainer-pin 249 that passes through a fasten-hole 226 in the working-blade 220 and then deforms the retainer-pin 249 by hot-press to mount the working-blade 220 to the fixing platform 239 on the handle 230 and firmly fixed. The handle 230 further includes a handle proximal-end 232 and a handle distal-end 238. One end of the boss 234 is connected with the handle proximal-end 232 of the handle and the other end is connected with the middle cylinder 236, which extends to the distal end and is connected with the handle distal-end 238, which extends to the distal end and connected with the fixing platform 239. The handle 230 further includes a handle axial-aperture 233 that includes a through-hole 235 that communicates with the handle axial-aperture 233. The through-hole 235 is approximately rectangular and includes a transverse short-side 235a and an axial long-side 235b, which is substantially parallel to the handle axial-aperture 233. The outer surface of handle proximal-end 232 includes a plurality of the first lock-catches 241, and the outer surface of the middle cylinder 236 includes a plurality of the second lock-catches 243.

Referring to FIG. 5 5-7, the fastened protector-shield 250 includes a proximal fastened-shield 252 and a slant distal fastened-shield 260 and a fastened-shield-base 254 therebetween. The fastened protector-shield 250 further includes an axis 251 (not shown) and a fastened-axial-aperture 253 that includes a proximal axial-aperture 256 and a distal axial-aperture 258. The proximal axial-aperture 256 extends from the proximal end through the proximal fastened-shield 252 and toward the distal end, the distal axial-aperture 258 extends from the distal end through the slant distal-end 260 and extends toward the proximal end. In the present embodiment, the inner diameter of the proximal axial-aperture 256 is larger than the inner diameter of the distal axial-aperture 258, and both of which intersect to form an inner step 257 that is substantially coaxial with the distal axial-aperture 258. The fastened protector-shield 250 further includes a plurality of fastened-shield lock-holes 255 that penetrate the proximal fastened-shield 252 and communicate with the proximal axial-aperture 256. The distal fastened-shield 260 includes a fastened slant outer-surface 262, and the distal axial-aperture 258 penetrates the fastened slant outer-surface 262 to form a fastened-shield-lip 266.

Those skilled in the art could appreciate that the fastened protector-shield 250 and the handle 230 can be held together by a variety of well-known joining techniques, such as bonding, welding, mechanical securing, and so on. In the present embodiment, the proximal axial-aperture 256 is matched with the shape and size of the middle cylinder 236, and the second lock-catch 243 is snapped into the fastened-shield lock-holes 255, thereby, the fastened protector-shield 250 and the handle 230 is firmly held together. Referring to FIG. 6 and FIG. 7, all or a portion of the blade-edges 224 is exposed outside the fastened protector-shield 250, i.e., all (or a portion) of the blade-edge 224 extends toward the distal end and beyond the fastened-shield-lip 266.

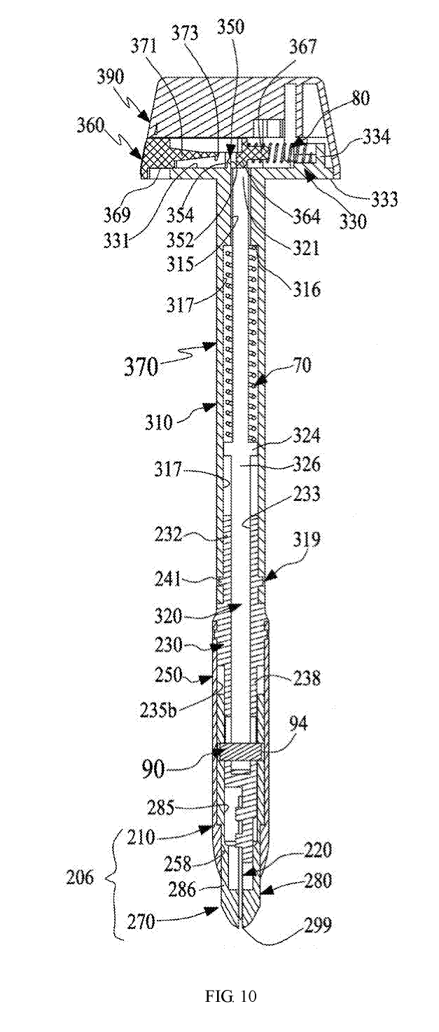

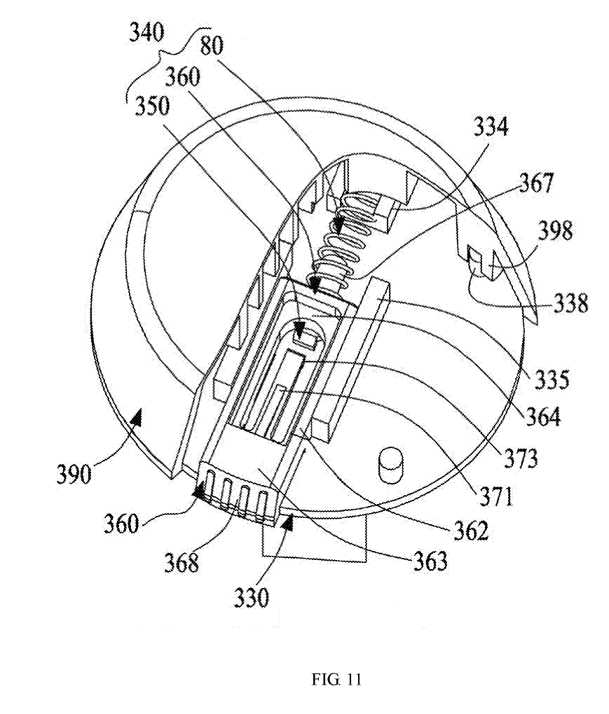

The shaft includes a fastened shaft 310 and a movable shaft 320. Referring to FIG. 5 and FIG. 10, the fastened shaft 310 includes a central axis 311 (not shown), a shaft proximal-end 312 and a shaft distal-end 318 and a connecting rod 314 therebetween. The fastened shaft 310 also includes a fastened shaft-aperture 313 that axially penetrates the shaft proximal-end 312 and the shaft distal-end 318. The fastened shaft-aperture 313 includes the first shaft-aperture 315 and the second shaft-aperture 317, the first shaft-aperture 315 penetrating from the proximal end to the shaft proximal-end 312 and extending toward the distal end, the second shaft-aperture 317 extends from the distal end through the shaft distal-end 318 toward the proximal end. In the present embodiment, the inner diameter of the first shaft-aperture 315 is smaller than the inner diameter of the second shaft-aperture 317, the two of which intersect to form the inner-shoulder 316. The fastened shaft 310 further includes a plurality of shank locking holes 319 that penetrate the distal end 318 transversely and communicate with the second shaft-aperture.

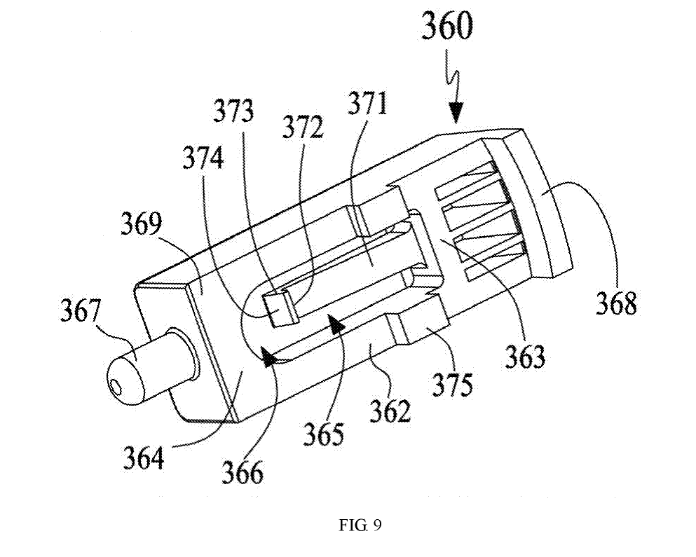

Referring to FIG. 5, FIG. 8 and FIG. 9, the movable protector-shield 280 includes a proximal movable-shield 282 and a movable slant distal-end 290 and a movable-shield-base 284 there between. The movable protector-shield 280 also includes a central shaft 281 and a movable axial-aperture 283. The movable axial-aperture 283 includes a proximal axial-aperture 285 and a distal blind-aperture 287, the proximal axial-aperture 285 extending from the proximal end through the proximal movable-shield 282 to the distal end and connected with the distal blind-aperture 287. In the present embodiment, the inner diameter of distal blind-aperture 287 is smaller than the inner diameter of the proximal axial-aperture 285, the two of which intersect to form a step 286. The distal blind-aperture 287 extends toward the distal end to the interior of the movable slant distal-end 290. The movable protector-shield 280 further includes a side-aperture 289 that transversely penetrates the proximal movable-shield 282 and communicates with the proximal through-hole 285. The movable slant distal-end 290 includes a movable slant outer-surface 292 and a groove 293 that transversely penetrates the movable slant outer-surface 292 to form a top end 299 and a sloped edge 298.

Referring to FIG. 5, the movable shaft 320 includes a proximal movable-shaft 322 and a distal movable-shaft 326 and a boss 324 there between, and the diameter of the boss 324 is larger than the diameter of the proximal movable-shaft 322. The proximal movable-shaft 322 includes a proximal shaft-head 321 that includes a distal shaft-head 327 that includes a lash-aperture 325.

Referring to FIGS. 5 and 10, the movable shaft 320 is mounted inside the handle 230 and the fastened shaft 310, wherein the proximal movable-shaft 322 is matched with the first axial-aperture 315, and the the distal movable-shaft 326 mates with the handle axial-aperture 233 and the lash-aperture 325 is substantially aligned with the through-hole 235. The movable protector-shield 280 is mounted inside the fastened protector-shield 250, wherein the movable-shield-base 286 matches the shape and size of the distal axial-aperture 258, and the proximal through-hole 285 matches the shape and size of the handle distal-end 238, and the side-aperture 289 and the lash-aperture 325 are aligned. The lash 90 includes a long side 92, a wide side 94 and a high side 96. The lash 90, the lash-aperture 325, the through-hole 235 and the side-aperture 289 are matched in shape and size, and the lash 90 passes through the side-aperture 289, the through-hole 235 and the lash-aperture 325 in order from the outside to the inside, and the lash 90 and the side-aperture 289 have the interference fit, thereby the movable protector-shield 280 and the movable shaft 320 are firmly fixed together. The dimension of the long side 235b in the axial direction is larger than the dimension of the wide side 94 of the lash 90, and the movable protector-shield 280 and the movable shaft 320 can move together in the axial direction. When the movable protector-shield 280 and the movable shaft 320 move from the distal end to the proximal end to expose the blade-tip 223 or the blade-edge 224, and the distal-end portion 206 is said to be in the operation mode; when the movable protector-shield 280 and the movable shaft 320 move from the proximal end to the distal end to cover the blade-tip 223 and the blade-edge 224 and are locked (i.e., the movable protector-shield 280 and the movable shaft 320 cannot move from the distal end to the proximal end), the distal-end portion 206 is said to be in the protection mode. The reset spring 70 of the protection shield is mounted on the proximal shaft 322 between the inner shoulder 316 and the boss 324 in a compressed state, that is, the reset spring 70 of the protection shield has an axial tension, thereby driving the movable shaft 320 and the movable protector-shield 280 to move from the proximal end to the distal end. In the natural state (i.e. the state in which the protection shield is not subjected to an external force), the movable shaft 320 and the movable protector-shield 280 automatically move to the distal end point of the axial movement stroke by the axial tension.

The handle 202 includes a handle base 330, a lock mechanism 340 and a handle housing 390. The handle base 330 includes a flange 332, which comprises handle top-surface 211 is connected with the handle under-surface 213. The flange 332 further includes a retainer-seat 334, a guide rib 335, a guide groove 336, a notch 337 and four approximately uniform retainer-pins 338. Referring to FIG. 5, the handle housing 390 includes a handle top-surface 391, a side-wall 392 and a button-notch 393. The handle housing 390 further includes four retainer-pins 398 with the central blind-aperture and a plurality of axial limit ribs (not shown). The lock mechanism 340 includes a lock-teeth 350, a lock member 360 and a lock-member reset spring 80. The lock-teeth 350 includes a lock surface 352 and a sloping surface 354. In an alternative embodiment, the fastened shaft 310, the handle base 330 and the lock-teeth 350 are joined together to form a single component, referred to as a main-body 370. The shaft proximal-end 312 is integrally connected with the handle bottom-surface 333, and the first axial-aperture 315 penetrates the flange 332. The lock-teeth 350 are integrally connected with the upper surface 331 of the flange 332, and the locking-surface 352 is circumscribed with the first axial-aperture 315.

The lock 360 has a proximal plane 361 and a distal plane 369. The lock 360 includes a release end 363 and a locking end 364. Two guide walls 362 join the release end 363 and the locking end 364 together to form an approximately rectangular cavity 365 that includes a semi-circular aperture 366 at the locking end 364. The locking end 364 includes a spring retainer-shaft 367. The release end 363 includes a button 368 and a trigger arm 371. The trigger arm 371 extends from the release end 363 toward the interior of the cavity 365, and the trigger arm 371 includes a release hook 373. The release hook 373 includes an occlusal surface 372 and a trigger surface 374. The distal plane 369 includes a guide block 375.

Referring to FIGS. 5-10, mainly referring to FIG. 10. The lock member 360 is mounted on the flange 332, wherein the guide wall 362 mates with the guide rib 335, and the distal plane 369 mates with the upper surface 331, so the lock member 360 is slidable along the guide rib 335 in a plane limited by the upper surface 331. One end of lock-member reset spring 80 is mounted in the retainer-seat 334, and the other end thereof is mounted on the spring retainer-shaft 3367, in a compressed state. Those skilled in the art could appreciate that the handle housing 390 can be secured on the handle base 330 by a variety of well-known joining techniques, such as bonding, welding, mechanical securing, and so on. In the present embodiment, four retainer-pins 338 are aligned with the central blind-aperture of the four retainer-pins 398 and have the interference fit, thereby the handle base 330 and the handle housing are 390 firmly fixed together, and a plurality of axial limit ribs respectively limit the axial displacement of the lock member 360 and the lock-member reset spring 80. Those skilled in the art can make a slight adaptation, and it is easy to understand and use the axial limit ribs to achieve the following functions: the lock member 360 can slide along the guide rib 335 in the plane limited by the upper surface 331 and its axial direction (direction of the parallel axis 201) is sufficiently small; the lock-member reset spring 80 is freely expandable and deformable and its axial direction (direction of the parallel axis 201) is sufficiently small. Due to space limitations and in order to simplify the description, the structure of the axial limit rib is not disclosed in detail in the illustration of the present invention.

The initial lock state: referring to FIGS. 9 and 10, the lock-member reset spring 80 is in a compressed state and has a transverse relaxation tension that urges the lock member 360 to slide along the guide rib 335 toward the outside of the handle housing 390 to the outer end point of the transverse motion; and the locking end 364 blocks the first axial-aperture 315, and the release hook 373 does not contact the lock-teeth 350, which is called a lock state. When in the locked state, the movable protector-shield 280 completely covers the working-blade 220 and is locked (i.e., the movable protector-shield 280 and the movable shaft 320 cannot be moved from the distal end to the proximal end), and the distal-end portion 206 of the obturator 200 is in the protection mode.

The release state: referring to FIG. 11, an external force is applied to press the button 368 to move the lock member 360 along the guide rib 335 toward the inside of the handle housing 390, and the lock-member reset spring 80 is continuously compressed until the trigger surface 374 of the release-hook 373 contacts the slant surface 354 of the lock-teeth 350; continuing to slide, the sloping surface 354 presses the trigger surface 374, so the trigger arm 371 is elastically deformed and the release hook 373 is axially displaced from the distal end to the proximal end; and continuing to slide, the release hook 373 spans the lock-teeth 350, and the trigger arm 371 rebounds, so the locking surface 352 meshes with the occlusal surface 372. At this time, the locking end 364 has been removed to expose the first axial-aperture 315, and the movable protector-shield 280 and the movable shaft 320 can be moved from the distal end to the proximal end, which is called a release state. When stopping to apply the external force, the relaxation tension of the lock-member reset spring 80 urges the lock member 360 to slide along the guide rib 335 toward the outside of the handle housing 390, and since the release hook 373 meshes with the lock tooth 350, the lock member 360 cannot slide and is in a stable state.

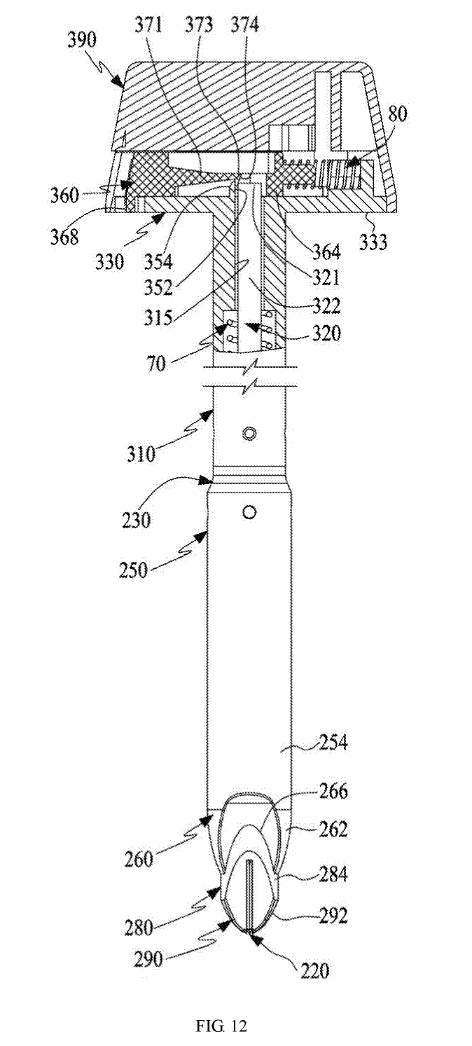

The trigger state: referring to FIGS. 3 and 4, the obturator 200 is inserted through the cannula assembly 100 and then together penetrate through the skin incision. Pressing the button 368 as described above causes the obturator 200 to be in the release state. Referring to FIG. 11, when the protection shield 280 is subjected to an axial compressive force, the protection shield 280 and the movable shaft 320 are moved from the distal end toward the proximal end to the blade-tip 223 and the blade-edge 224 of the working-blade 220 are exposed. State 1, referring to FIG. 11, the proximal shaft-head 321 of the movable shaft 320 contacts the trigger surface 374 of the release hook 373, and continued continues to move and force the trigger arm 371 deformed and the release hook 373 to produce the axial displacement from the distal end to the proximal end to disengaged from the lock-teeth 350, that is, the lock member is released; state 2, referring to FIG. 12, the proximal shaft-head 321 continues to move from the distal end to the proximal end of the axial movement stroke, at which point the release hook 373 has been completely disengaged from the lock-teeth 350, the lock member 360 slides along the guide rib 335 toward the outside of the handle housing 390 under the action of lock-member reset spring 80 until the locking end 364 is blocked by the proximal movable-shaft 322; the distal-end portion 206 of the obturator 200 in the state 1 and state 2 is in the working mode.

The reset lock state: once the obturator 200 completely penetrates into the body wall, the transverse pressure and the axial resistance of the movable protector-shield 280 disappear, and the movable protector-shield 280 and the movable shaft 320 rapidly move under the shield reset-spring 70 toward the distal end to the proximal ending point of the axial movement stroke. While the lock member 360 rapidly slides along the guiding rib 335 toward the outer direction of the handle housing 390 under the thrust of the lock-member reset spring 80 until the locking end 364 blocks the first axial-aperture 315, so that the proximal shaft-head 321 cannot be retracted from the distal end to the proximal end, and the distal-end portion 206 of the obturator is transformed from the operation mode to the protection mode. That is, when the obturator continues to move toward the body cavity and contacts the organ or tissue in the cavity, the blade-tip 223 and the blade-edge 224 are not exposed, and only the movable protector-shield 280 contacts the organ or tissue in the cavity

In the present embodiment, the lock mechanism 340 includes a lock-teeth 350, a lock member 360 and a lock-member reset spring 80, and realize the switch between working mode and protection mode. However, the lock mechanism 340 can be implemented in a variety of ways. Since the first protection obturator has been disclosed in U.S. Pat. No. 4,535,773, the designers have successively disclosed a large number of the lock mechanism for achieving mutually switch between the protection state (i.e. the protection shield of the protector is locked) and the release state (i.e. the protection shield of the protector is movable) of the protection obturator. Those skilled in the art will readily appreciate that simple adaptations to the disclosed lock mechanism can be used to switch between the operation mode and the protection mode of the present invention. Other similar lock mechanisms are also conceivable to those skilled in the art.

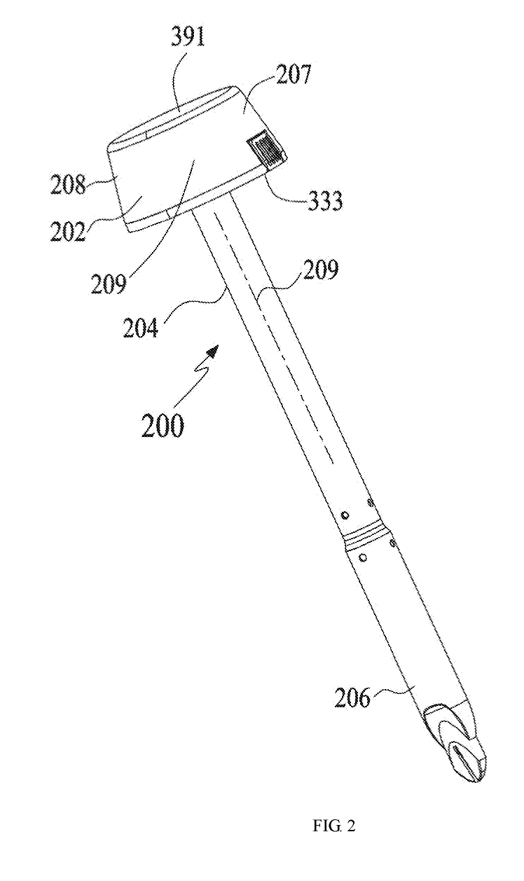

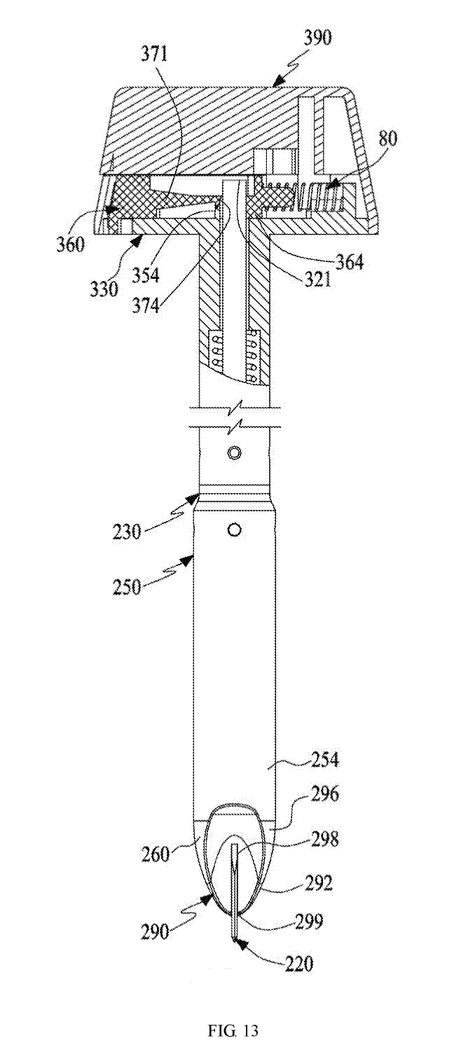

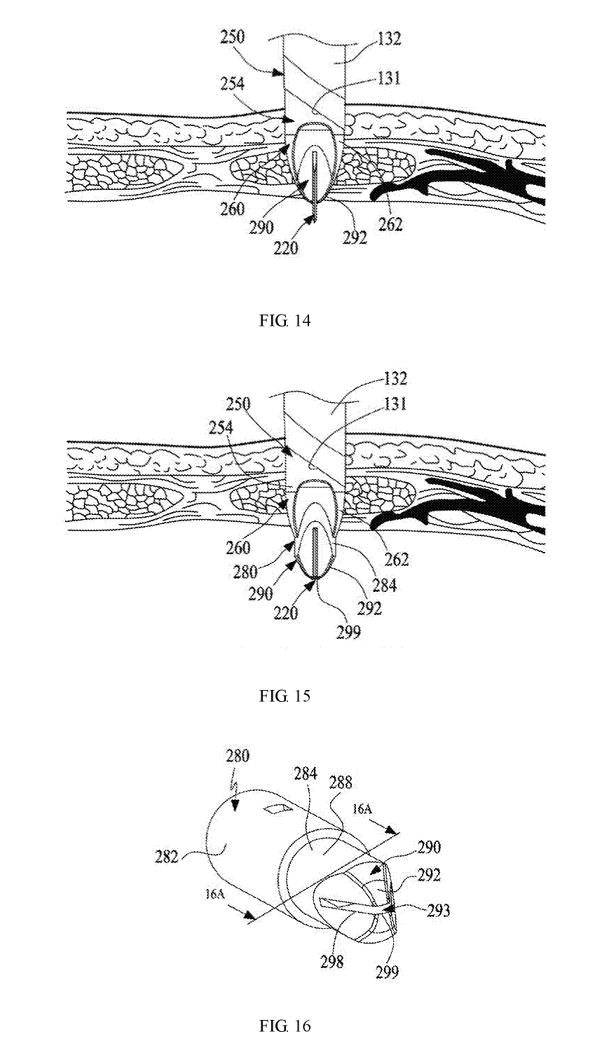

Features and applications of the trocar 1000: referring to FIG. 2 and FIG. 12, in one aspect of the invention, the obturator 200 extends through the cannula 100 when the handle bottom-surface 333 contacts the cannula top-surface 111, the fastened shield of the fastened protector-shield 250 has a slant distal-end 260 that extends to the distal end beyond the sleeve-lip 131. As described above, pressing the button 368 causes the distal-end portion 206 to be in a released state and then penetrate together into the body through the skin incision at the penetration point. Referring to FIGS. 12-13, during the penetration into the body, the patient's muscle imparts resistance to the movable protector-shield 280 from the body to the outside, and the movable protector-shield 280 is moved together with the movable shaft 320 from the distal end to the proximal end to exposed blade-tip 223 and blade-edge 224; at the same time, the proximal shaft-head 321 triggers the lock mechanism 340 to cause the distal-end portion 206 to transform from the release state to the trigger state. When in the trigger state, the movable-shield-base 284 is fully or mostly retracted into the interior of the distal axial-aperture 258, and the movable slant outer-surface 292 and the fastened slant outer-surface 262 have the smooth transition, so that the overall shape of the distal-end portion 206 is streamlined to facilitate penetrating, tearing, expanding and swelling the muscles and tissue.

The process of penetrating the body wall can be divided into two stages: the first stage, from the beginning of the penetration to the blade-edge 234 and the movable slant distal-end 290 penetrating the body wall; the second stage, from the blade-edge 234 and the movable slant distal-end 290 penetrating the body wall to the distal-end 132 of the cannula penetrating the body wall. The main work of the first stage penetration includes that the blade-edge 234 penetrates and cuts muscle tissue, the movable slant distal-end 290 tears and expands muscle tissue, and the fastened slant distal-end 260 and the distal-end 132 of the cannula expand (swell) muscle tissue. The main work of the second stage penetration includes that the fastened slant distal-end 260 and the distal-end 132 of the cannula expand (swell) muscle tissue. Although the trocar 10 described in the background includes only one protection shield, when the trocar 10 is used for penetration, the main work process can still be approximated into two stages, but the surgeon does not feel the obvious boundary. Those skilled should understand in the art that during the penetration process, different patient, or different location of the same patient, or different operation methods of the same location may cause different distance between the body wall and the interior organs. Therefore, it is very dangerous for the blade 234 to be exposed outside the protection shield during the second stage of penetration. Moreover, since the blade 234 is no longer working at the second stage of penetration, it is completely unnecessary to be exposed outside the protection shield, and it is also unreasonable.

The trocar 1000 of the present invention, at the second stage of penetration, that is, when the blade-edge 234 and the movable slant distal-end 290 penetrate into the body wall, since all or most of the movable-shield-base 284 is retracted inside the distal axial-aperture 258, there is no frictional resistance (or only a small frictional resistance) between the movable protector-shield 280 and the body wall muscle, and the movable protector-shield 280 can be quickly reset by the shield reset spring 70. That is, the movable protector-shield 280 and the movable shaft 320 are rapidly moved toward the distal end to the under the thrust of the lock reset spring 70 until the locking end 364 blocks the first axial-aperture 315, so that the proximal shaft-head 321 cannot be retracted from the distal end to the proximal end, and the distal-end portion 206 of the obturator is transitioned from the working mode to the protection mode. During the puncture, the trocar 1000 facilitates reducing the distance the blade 234 exposed within the body cavity and reducing the time the blade 234 exposed within the body cavity.

During the penetration, the ratio of the first stage and the second stage (hereinafter referred to as the ratio), that is, the reset timing of the movable protector-shield 280, has a significant influence on the use performance of the trocar. If the ratio of the first stage penetration is too small, the amount of penetration and tear will be usually insufficient, which inevitably leads that the amount of penetration and expansion in the second stage is too large, thereby increasing the penetration force and reducing the controllability of the penetration operation. If the penetration ratio of the first stage is too large, that is, the amount of penetration and tear is too large, the penetration workload of the first stage will be too large or the working-blade 220 exposed into the body is too long, thereby reducing the safety of the penetration operation. Reasonably setting the interface size of the movable protector-shield 280 and the fastened protector-shield 250, that is, properly setting the size of the movable-shield-base 284, in the present embodiment, can effectively control the ratio of the first penetration stage and the second penetration stage.

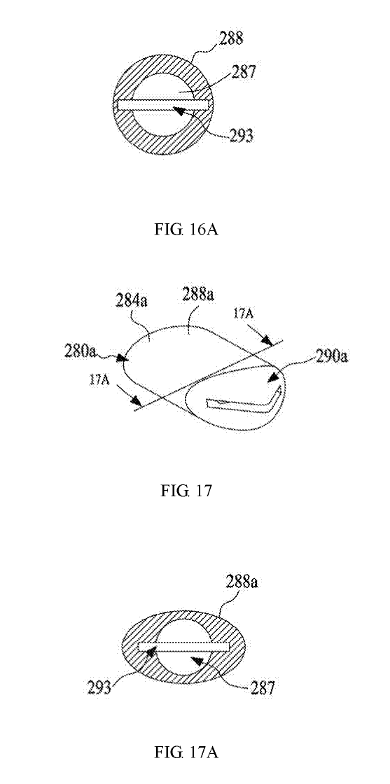

Referring to FIG. 16 and FIG. 16A, the movable-shield-base 284 includes a cylindrical surface 288, that is, the movable cover base 284 is a cylindrical-shape as a whole. Optionally, the geometric relationship of the movable-shield-base 284, herein conforms to the following equation:

0.5D₀≤D≤0.85D₀

0.25A₀≤A≤0.72A₀

wherein:

D=the outer diameter of the movable-shield-base (that is the diameter of the cylinder limited by the cylindrical surface 288);

D₀=the maximum outer diameter of the distal-end portion 206 of the obturator;

A=the cross-section area limited by the cylindrical surface 288 (ignoring the structure of the internal hollow groove);

A₀=the cross-sectional area at the maximum outer diameter of the distal-end portion 206 of the obturator (ignoring the structure of the internal hollow groove).

Those skilled in the art should understand that the length of the distal-end portion 206 of the obturator absolutely exceeds the total length of the distal end 132 of the cannula, usually between 15 mm-25 mm, which is limited by the body wall and the body cavity structure and the clinical application. When the outer diameter of movable-shield-base 284 is smaller than half of the maximum outer diameter of the distal-end portion 206, the amount of puncturing and tearing of the cutting edge 224 and the movable cover inclined distal end 290 is too small, resulting in at the second stage of the penetration, the fastened slant distal-end 260 and the distal end 132 of the cannula are expanded and swelled by a large amount, thereby increasing the penetration force and reducing the controllability of the penetration operation. When the outer diameter of the base 284 is 0.85 times larger than the maximum outer diameter of the distal portion 206, or the angle of inclination of the movable inclined distal end 290 from the distal end to the proximal end is too large (i.e. the transverse dimension of the slant distal-end 290 growth rate is excessively increased), resulting in a large penetration and tearing force; or causing the blade 224 to be exposed to an excessively large depth, thereby reducing the safety of the penetration operation. Those skilled should understand in the art that when the cross section is circular, the aforementioned area formula and diameter formula are equivalent.

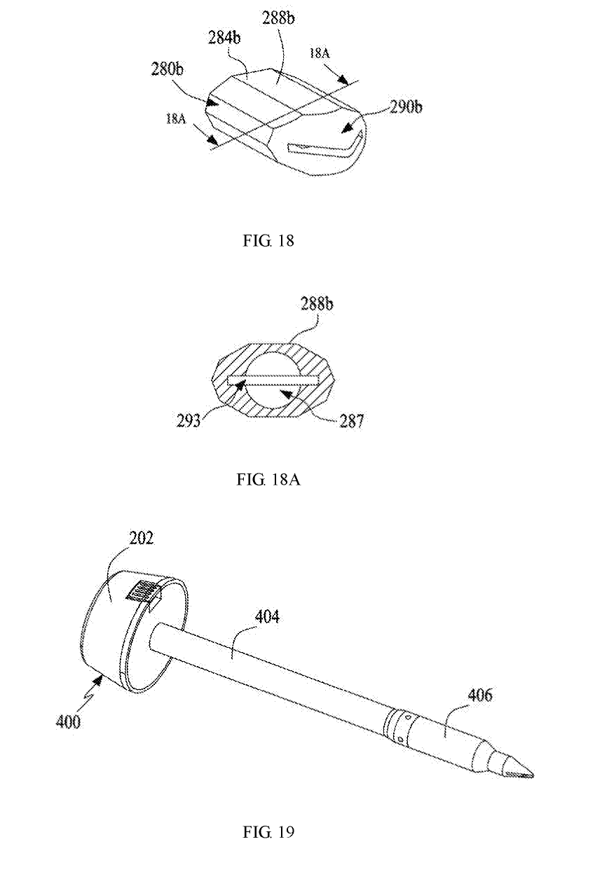

Although the movable-shield-base 284 is a cylinder with the circular cross-section in the embodiment that has been shown, the movable-shield-base 284 may also be a cylinder with the oval or the arbitrary polygonal cross-section. Referring to FIG. 17 and FIG. 17A, in one solution, the movable protector-shield 280a includes a movable-shield-base 284a and a movable slant distal-end 290a. The movable-shield-base 284a includes a straight cylindrical surface 288a (a straight cylindrical surface, that is, a curved surface formed by the straight bus moving parallel along a fixed curve), and the straight cylinder surface 288a is an elliptical cylinder formed by a straight bus around the ellipse moving parallel, and the movable-shield-base 284a is an elliptical straight cylinder. The straight cylindrical surface 288a is substantially perpendicular to the cross section and its cross section is an elliptical shape. Referring to FIG. 18 and FIG. 18A, in another solution, the movable protector-shield 280b includes a movable protector-shield 284b and a movable slant distal-end 290b. The movable-shield-base 284b includes a straight cylindrical surface 288b, which is a polygonal cylindrical surface formed by a straight bus around the polygon moving parallel, and the protection shield 284b is a polygonal straight cylinder. The straight cylindrical surface 288b is substantially perpendicular to the cross section and its cross section is a polygon. Those skilled in the art will appreciate that the distal axial-aperture 258 should be shaped and sized to match the movable-shield-base 284. The outer surface of the movable-shield-base 284 may have a small taper for ease of manufacture (e.g. ease of demolding), preferably, when the taper should be small enough and the movable protector-shield 280 is at the distal endpoint and the proximal endpoint of the axial movement stroke, the one-side fit clearance between movable-shield-base 284 and the distal axial-aperture 258 is less than or equal to 0.5 mm. The surface of the movable-shield-base 284 may also include dimples or grooves for reducing the contact area or storing lubricating fluid to reduce the friction between the movable-shield-base 284 and the distal axial-aperture 258.

Referring to FIG. 17A and FIG. 18A, those skilled readily understand by those skilled in the art that when the movable-shield-base is non-cylindrical (e.g. the movable-shield-base 284a and the movable-shield-base 284b), the aforementioned diameter equation has not been applied to describe the size of the movable-shield-base and the relationship of the first stage penetration and the second one, however the aforementioned area equation still applies.

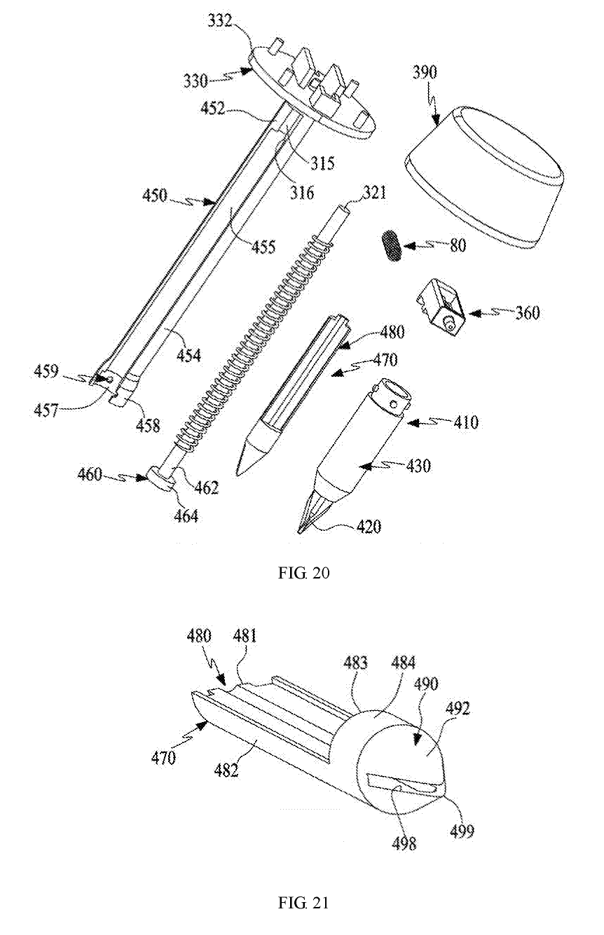

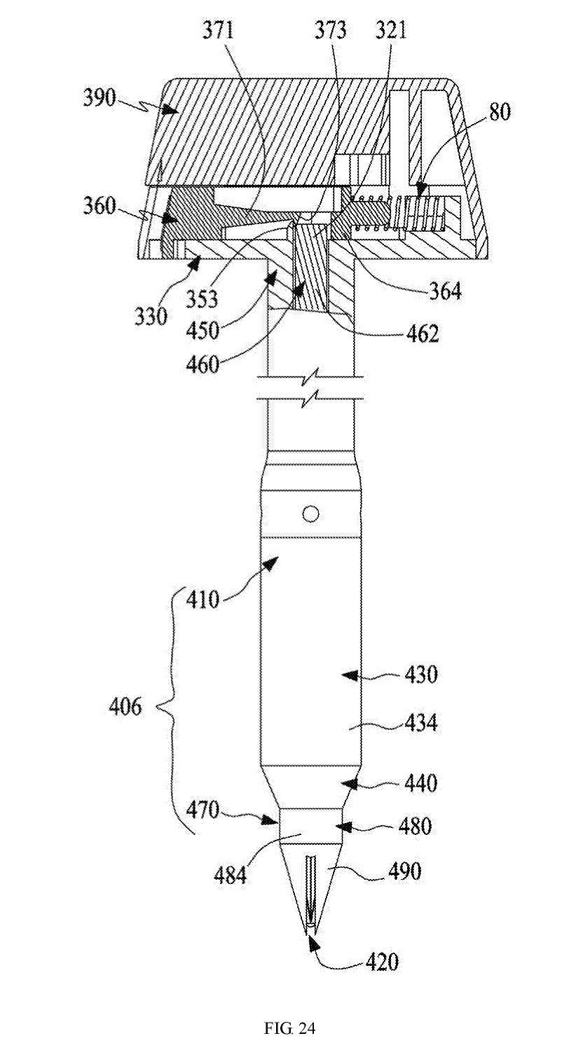

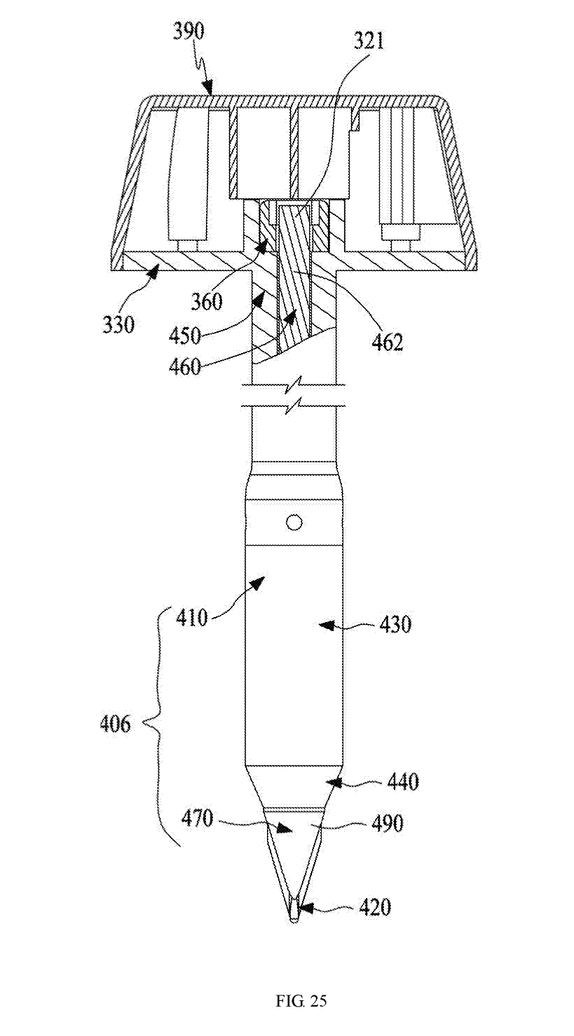

FIGS. 19-25 show detailed depiction the second embodiment in the invention, the composition and assembly relationship of the bladeless auto-protection obturator 400. The numerical designations of the geometrical structure in FIGS. 19-25 are the same as which in FIGS. 4-10, it indicates that the structure of the same designations of the embodiment 1 and the embodiment 2 is basically equivalent. Referring to FIGS. 19-20, the obturator 400 comprises a handle 202, a shaft portion 404 and a distal-end portion 406. The distal portion 406 comprises a fastened distal-portion 410 and a movable distal-portion 470. The fastened distal-portion 410 includes a plastic blade 420 and a fastened protection shield 430, and the movable distal-portion 470 includes a movable protection shield 480. The shaft 404 includes a fastened shaft 450 and a movable shaft 320. The handle in the obturator 400 and the one in the obturator 200 are completely identical, and details are not described herein.

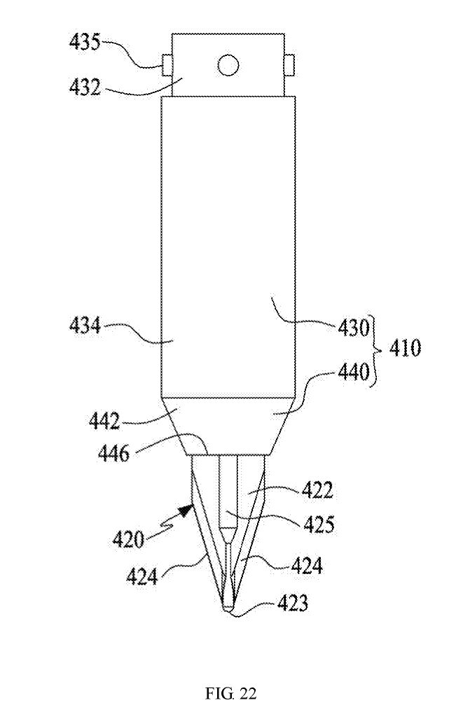

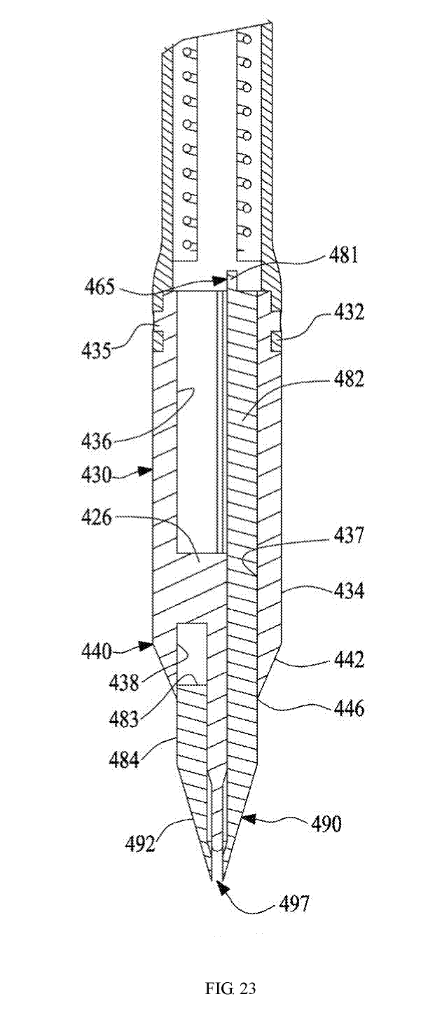

Referring to FIG. 22-23, the plastic blade 420 includes a flat blade-body 422, a blade-tip 423 and two blade-edges 425 forming an acute angle with each other. The plastic blade 420 further includes a blade reinforcing rib 425. Those skilled in the art could appreciate that the plastic blade 420, the fastened shaft 450 and the fastened protector-shield 430 can be secured together by a variety of well-known joining techniques, such as bonding, welding, mechanical securing, and so on. In the present embodiment, the plastic blade 420 and the protection shield 430 are integrally joined by the connecting-piece 426 to form a single injection molded part.

Referring to FIG. 22-23, the fastened protector-shield 430 includes a proximal fastened-shield 432 and a slant distal fastened-shield 440 and a fastened-shield-base 434 therebetween. The fastened protector-shield 430 further includes an axis 431 (not shown) and a fastened-axial-aperture 433 that is divided by the connecting-piece 426 into three parts including a proximal axial-aperture 436, a transitional axial-aperture 437 and a distal axial-aperture 438. The proximal fastened-shield 432 includes a plurality of third lock-catche s 435. The slant distal-end 440 includes a slant outer surface 442, and the distal axial-aperture 438 penetrates the outer surface 442 to form a fastened-shield-lip 446.

Referring to FIG. 21 and FIG. 23, the movable protector-shield 480 includes a proximal movable-shield 482 and a movable slant distal-end 490 and a movable-shield-base 484 therebetween. The proximal movable-shield 482 includes a proximal connecting-axial 481 that forms a step 483 at the intersection of the proximal movable-shield 482 and the movable-shield-base 484. The movable slant distal-end 490 includes a movable slant outer-surface 492 and a groove 493 that transversely penetrates the movable slant outer-surface 492 to form a top end 499 and a sloped edge 498.

Referring to FIG. 20 and FIG. 23, the fastened shaft 450 includes a central axis 451 (not shown), a shaft proximal-end 452 and a shaft distal-end 458 and a connecting rod 454 therebetween. The fastened shaft 450 also includes a fastened axial-aperture 453 that axially penetrates the shaft proximal-end 452 and the shaft distal-end 458. The fastened axial-aperture 353 includes the first axial-aperture 315 and the third axial-aperture 455, and the fourth axial-aperture 457, the first axial-aperture 315 penetrating from the proximal end to the shaft proximal-end 452 and extending toward the distal end, the fourth shaft-aperture 457 extends from the distal end through the shaft distal-end 458 toward the proximal end. The diameter of the first axial-aperture 455 is smaller than the inner diameter of the third axial-aperture 315, the two of which intersect to form the inner-shoulder 316. The fastened shaft 450 further includes a plurality of shank locking holes 459 that penetrate the distal end 458 transversely and communicate with the fourth axial-aperture. The fastened shaft 450 includes a handle base 330, the shaft proximal-end 452 is integrally connected with the Hang 332 of the handle bottom-surface 330, and the first axial-aperture 315 penetrates the flange 332. The movable shaft 460 includes a proximal movable-shaft 462 and a boss 464, the diameter of which is larger than the diameter of the proximal movable-shaft 462. The proximal movable-shaft 462 includes a proximal shaft-head 321, and the boss 464 includes a connecting-groove 465.

Those skilled in the art could appreciate that the fastened protector-shield 410 and the fastened shaft 450 can be held together by a variety of well-known joining techniques, such as bonding, welding, mechanical securing, and so on. Referring to FIG. 23, in the present embodiment, the proximal fastened-shield 432 is matched with the shape and size of the fourth axial-aperture 457, and the third lock-catch 435 is snapped into the fastened-shield lock-holes 459, thereby, the fastened protector-shield 410 and the handle 450 is firmly held together. The movable protector-shield 480 is mounted inside the fastened protector-shield 410, wherein the movable-shield-base 484 matches the shape and size of the distal axial-aperture 438, and the proximal blade-body 422 matches the shape and size of the blade-groove 497. The movable shaft 460 is mounted to the interior of the fastened shaft 450, wherein the proximal movable-shaft 462 is matched with the first axial-aperture 315, and the connecting-groove 465 is matched with the connecting-axial 481. The connecting-axial 481 and the connecting-groove 465 can be secured to each other by various known connecting techniques. This embodiment adopts a glue bonding method to firmly secure the movable shaft 460 and the movable protector-shield 480 together.

The reset spring 70 of the protection shield is mounted on the proximal movable-shaft 462 between the inner shoulder 316 and the boss 464 in a compressed state, that is, the reset spring 70 of the protection shield has an axial tension, thereby driving the movable shaft 460 and the movable protector-shield 480 to move from the proximal end to the distal end. In the natural state (i.e. the state in which the protection shield is not subjected to an external force), the movable shaft 460 and the movable protector-shield 480 automatically move to the distal end point of the axial movement stroke by the axial tension (when the boss 464 is contacted with the proximal fastened shield 432). Likewise, when the movable protector-shield 480 and the movable shaft 460 move from the distal end to the proximal end to expose the blade-tip 423 or the blade-edge 424, and the distal-end portion 406 is said to be in the operation mode; when the movable protector-shield 480 and the movable shaft 460 move from the proximal end to the distal end to cover the blade-tip 423 and the blade-edge 424 and are locked (i.e. the movable protector-shield 480 and the movable shaft 460 cannot move from the distal end to the proximal end), the distal-end portion 406 is said to be in the protection mode.

Referring to FIGS. 19, 24 and 25, all of the obturator 400 and the obturator 200 comprise an equivalent handle portion, that is, there comprise an equivalent handle base 330, a lock mechanism 340 and a handle housing 390. Therefore, the obturator 400 includes equivalently an initial lock state, a release state, a trigger state, and a reset lock state. That is, the obturator 400 has a function substantially equivalent to that of the obturator 200, and the main bladeless obturator, and the obturator 200 belongs to a blade obturator. Both types of obturators have different degrees of damage to the patient, and the penetration force required during the penetration is different.

The invention has repeatedly mentioned the concept of the blade automatic protection obturator and a bladeless automatic protection obturator. It should be easily understood by those skilled in the art that the obturator used in endoscopic surgery can be generally divided into two types: a blade obturator and a bladeless obturator. The “blade” refers to a metal-blade, and the “bladeless” refers to a metal-free blade. An obturator with a plastic blade is often referred to as a bladeless obturator, which is the convention in the art. The blade and bladeless generally represent the damage degree of the obturator against the muscles. As mentioned in the background, the operation mode of the blade obturator is usually mainly to penetrate and cut the muscles, while the operation mode of the bladeless obturator is usually mainly to penetrate and tear the muscle. The blade obturator has a relatively large degree of damage to the muscle, and its penetration force is relatively small. For convenience of description, the metal blade or plastic blade of the working-blade is used in the present invention.

]In the first embodiment and the second embodiment of the present disclosure, the working-blade and the fastened protector-shield or fastened shaft are connected together, but the working-blade can also be fixed to the handle by the elongated shaft. U.S. Pat. No. 6,319,266 discloses a solution in which the working-blade is coupled to the handle housing by the elongated shaft. A slight modification to the first embodiment and the second embodiment can also realize that the working-blade is not connected with the fastened protector-shield or the fastened shaft, and is connected with the handle base or the handle housing.

Many different embodiments and examples of the invention have been shown and described. One ordinary skilled in the art will be able to make adaptations to the methods and apparatus by appropriate modifications without departing from the scope of the invention. For example, the lock mechanism and the connection mechanism disclosed in other inventions, or the locking structure and the limiting structure are adaptively modified, or the external shape of the distal half is modified, or a shrapnel is used instead of the spring, and so on. Several modifications have been mentioned, to those skilled in the art, other modifications are also conceivable. Therefore, the scope of the invention should follow the additional claims, and at the same time, it should not be understood that it is limited by the specification of the structure, material or behavior illustrated and documented in the description and drawings.

What is claimed is:

1. A trocar comprising an obturator and a cannula; the cannula including a hollow sleeve which including a sleeve distal-end and a sleeve-lip; the obturator including a handle, the distal-end portion and a shaft there between; wherein the distal-end portion includes a fastened protection shield, a working-blade and a movable protection shield; the shaft includes a fastened shaft and a movable shaft, the fastened protection shield comprises a base of the fastened shield and a slant distal-end of the fastened shield connected thereto; the slant distal-end including a distal shaft-aperture which penetrates the slant distal-end to form a fastened-shield-lip; the fastened protection shield is connected with the fastened shaft, the working-blade extending toward the proximal end and connected with a fastened shaft or handle; the working-blade extending toward the distal end beyond the fastened-shield-lip.

2. The trocar of claim 1, when passing the obturator through the cannula for the penetration, the slant distal-end of the fastened shield is exposed outside the sleeve-lip, and the movable protection shield comprises a base of the movable shield and the slant distal-end of the movable shield connected thereto.

3. The trocar of claim 2, the distal-end portion includes an operating mode and a protection mode, in the operating mode, the movable protection shield is movable from the distal end to the proximal end to expose the working-blade, and in the protection mode, and the working-blade is covered by the movable protection shield; the handle includes a lock mechanism to effect mutual switching between the working mode and the protection mod.

4. The trocar of claim 3, wherein the base of the movable shield comprises a straight cylinder formed by the straight cylinder-surface, the shape and size of the straight cylinder matching the distal axial-aperture.

5. The trocar of claim 3, wherein the size of the movable-shield-base conforms to the following equation:
0.25A₀≤A≤0.72A₀
wherein:
A=cross-sectional area of the straight cylinder,
A₀=the maximum cross-sectional area of the distal-end portion.

6. The trocar of claim 1, wherein the working-blade is a metal blade or a plastic blade.

7. The trocar of claim 1, wherein the handle of the obturator includes a lock mechanism, which comprises at least a lock, a release, and a trigger.
